(19) United States
(12) Patent Application Publication
(10) Pub. No.: US 2009/0024211 A1
(43) Pub. Date: Jan. 22, 2009

(54) STENT WITH A COATING OR FILLING OF A CAVITY

(75) Inventor: Eric Wittchow, Nuernberg (DE)

Correspondence Address: POWELL GOLDSTEIN LLP, ONE ATLANTIC CENTER FOURTEENTH FLOOR, 1201 WEST PEACHTREE STREET NW, ATLANTA, GA, 30309-3488, US

(73) Assignee: Biotronik VI Patent AG, Baar (CH)

(21) Appl. No.: 12/171,536

(22) Filed: Jul. 11, 2008

(30) Foreign Application Priority Data
Jul. 20, 2007 (DE) 10 2007 034 019.4

(51) Int. Cl. A61F 2/82; A61L 33/00

(52) U.S. Cl. 623/1.45; 427/2.24

(57) ABSTRACT

A stent of a metallic base body and with an SiO₂-containing or silicate-containing coating or filling of a cavity.

### PRIORITY CLAIM AND CROSS REFERENCE TO RELATED APPLICATION

This patent application claims priority to German Patent Application No. 10 2007 034 019.4, filed Jul. 20, 2007, the disclosure of which is incorporated herein by reference in its entirety. This application is related to co-pending U.S. patent application Ser. No. ______, Attorney Docket No. 149459.00036, filed Jul. 11, 2008, and entitled Stent With A Coating, the disclosure of which is incorporated herein by reference in its entirety.

### FIELD

The present disclosure relates to a stent comprising a metallic base body and a coating or filling of a cavity as well as a method for manufacturing a stent.

### BACKGROUND

Implantation of stents has become established as one of the most effective therapeutic measures for treatment of vascular disorders. Stents have the purpose of assuming a supporting function in hollow organs of a patient. Stents of the traditional design, therefore, have a filigree supporting structure of metallic struts which are initially in a compressed form for introducing them into the body and are then widened at the site of application. One of the main areas of application of such stents is for permanently or temporarily widening vascular stenoses and keeping them open, in particular, constrictions (stenoses) of the myocardial vessels. In addition, there are also known aneurysm stents which serve to support damaged vascular walls.

Stents have a circumferential wall of adequate supporting force to keep the constricted blood vessel open to the desired extent and a tubular base body through which blood continues to flow unhindered. The supporting circumferential wall is usually formed by a mesh-like supporting structure which makes it possible to insert the stent in a compressed state with a small outside diameter up to the stenosis to be treated in the respective blood vessel and to widen the blood vessel there, e.g., with the help of a balloon catheter, so that the blood vessel has the desired enlarged inside diameter.

The stent has a base body of an implant material. An implant material is a non-living material that is used for an application in medicine and interacts with biological systems. The basic prerequisite for use of a material as an implant material which is in contact with the bioenvironment when used as intended is its biological compatibility (biocompatibility). For purposes of the present disclosure, biocompatibility is the ability of a material to induce an appropriate tissue response in a specific application. This includes adaptation of the chemical, physical, biological and morphological surface properties of an implant to the recipient tissue with the goal of a clinically desired interaction. The biocompatibility of the implant material also depends on the chronological course of the reaction of the biosystem into which the stent is implanted. Thus relatively brief irritation and inflammation occur and may lead to tissue changes. Biological systems thus react in various ways as a function of the properties of the implant material. According to the reaction of the biosystem, the implant materials may be subdivided into bioactive, bioinert and degradable/absorbable materials.

For the purposes of the present disclosure, only metallic implant materials are of interest for stents. Biocompatible metals and metal alloys for permanent implants include stainless steels (e.g., 316L), cobalt master alloys (e.g., L605, CoCrMo casting alloys, CoCrMo forge alloys, CoCrWNi forge alloys and CoCrNiMo forge alloys), pure titanium and titanium alloys (e.g., cp titanium, TiAl6V4 or TiAl6Nb7) and gold alloys. The use of magnesium or pure iron and biocorridible master alloys of the elements magnesium, iron, zinc, molybdenum and tungsten is proposed in the area of biocorridible stents.

A biological reaction to metallic elements depends on the concentration, exposure time and how supplied. The presence of an implant material frequently leads to inflammation reactions, but the triggering factors may be mechanical irritation, chemical substances or metabolites. The inflammation process is usually followed by infiltration of neutrophilic granulocytes and monocytes through the vascular walls, infiltration of lymphocyte effector cells with the formation of specific antibodies to the inflammation stimulus, activation of the complement system with the release of complement factors, which act as mediators, and ultimately the activation of blood coagulation. An immunological reaction is usually closely associated with the inflammation reaction and may lead to sensitization and development of an allergization. Known metallic allergens include, for example, nickel, chromium and cobalt, which are also used as alloy components in many surgical implants. An important problem in implantation of stents in blood vessels is in-stent restenosis due to overshooting neointimal growth, which is caused by great proliferation of the smooth muscle cells of the arteries and by a chronic inflammation reaction.

It is known that a higher measure of biocompatibility and thus an improvement in the restenosis rate can be achieved when metallic implant materials are provided with coatings of especially tissue-compatible materials. These materials are usually of an organic nature or a synthetic polymer nature and are partially of natural origin. Previous strategies to prevent restenosis have usually concentrated on inhibiting proliferation through medication, e.g., treatment with cytostatics.

Despite the progress that has been made, there is still a high demand for achieving a better integration of the stent into its biological environment and thereby decreasing the restenosis rate.

### SUMMARY

The present disclosure describes several exemplary embodiments of the present invention.

One aspect of the present disclosure provides a stent, comprising (i) a metallic base body; and (ii) either an SiO₂-containing or silicate-containing coating or an SiO₂-containing or silicate-containing filling of a cavity.

Another aspect of the present disclosure provides a method for manufacturing an SiO₂-containing coating on a stent of a metallic base body, comprising (i) providing the stent of a metallic base body; (ii) contacting a surface of the base body with an aqueous colloidal dispersion containing amorphous SiO₂ particles with an average particle size in the range of 5-75 nm; and (iii) either simultaneously or in following step (ii), thermally treating the stent at least in the area of the contact surface, forming the SiO₂-containing coating.

### DETAILED DESCRIPTION

According to a first aspect of the present disclosure, one or more of the problems described above are solved by a stent comprising a metallic base body and an SiO₂-containing or silicate-containing coating or filling of a cavity.

The present disclosure is based on the finding that in a healthy body there is an equilibrium between cell reproduction (cell proliferation) and cell death (apoptosis). If a restenosis occurs after implantation of a stent, the equilibrium between the two processes is disturbed and proliferation gains the upper hand over natural cell death. Previous strategies for preventing restenosis have been aimed at inhibiting proliferation. However, histological preparations of stenosed vessels have not shown elevated levels of proliferation markers in comparison with the surrounding tissue. This supports the assumption that apoptosis occurs less effectively than in healthy tissue. This is where the present invention begins. This imbalance is to be equalized by increasing the apoptosis rate. The advantage in comparison with inhibited proliferation is, among other things, that an accumulation of neointimal cells is prevented without delaying the required tissue coverage of the stent.

It has now surprisingly been found that the use of silicon dioxide or silicates as components of a coating or filling of a cavity of a metallic stent leads to increased apoptosis. The use of these inorganic substances has the special advantage that a high adhesion to the metallic base body can be achieved and the inorganic compounds have good thermal stability and are less reactive, so that the production and sterilization of the stent are simplified. The positive influence of silicon dioxide and silicates on the mechanism of action on which apoptosis is based remains largely unexplained. Presumably the capase-3 enzyme, which directly triggers the apoptotic process, is activated via a change in mitochondrial permeability.

For purposes of the present disclosure, silicon dioxide is the collective term for chemical compounds having the empirical formula SiO₂. For the purposes of the present disclosure, however, the crystalline modification of SiO₂ is evidently of only subordinate importance. The property of the material of accelerating apoptotic processes in biological systems is essential here.

The term silicate stands for a compound of silicon and oxygen with one or more metals and possibly also hydroxyl ions. Here again, according to preliminary investigations, the crystalline modification and the choice of the metal and/or the presence of hydroxyl ions are of subordinate importance with respect to the desired biological effect.

The apoptosis-stimulating material may be part of a coating or the coating may consist entirely of the material. The coating may be applied directly to the base body of the stent or additional layers in between may be provided. Alternatively, the apoptosis-stimulating material may be part of a cavity filling. The cavity is usually at the surface of the stent. In the case of stents having a biodegradable base body, the cavity may also be situated in the interior of the base body, so that the material is released only after being exposed. The coating or filling preferably contains 0.1 to 10 μg free or bound silicon per 1 mm stent length.

According to a first exemplary embodiment of the present disclosure, the coating or filling contains nanoparticles of SiO₂ or silicate. The nanopaiticles preferably have a particle size in the range of 5-75 nm and may be embedded in a polymer matrix, in particular, a polymer matrix containing polyurethane. The use of nanoparticles seems to be especially suitable for the inventive purposes due to the very large surface of the particles which is available for an interaction with the biological system.

In a second exemplary embodiment of the present disclosure, the coating is a closed film of SiO₂ or silicate. The film of SiO₂ or silicate may have a thickness in the range of 1 to 15 μm. According to this preferred embodiment, the metallic base body of the stent is thus applied directly or, if necessary, via additional intermediate layers so that it covers the surface of the stent. In the case of corrodible implants, a delay in degradation which is usually desired can be expected due to the relatively inert coating. During implantation of the stent, microcracks develop in the coating so that nothing stands in the way of degradation of the base body in the expanded state of the stent.

According to another exemplary embodiment, which can be implemented with the two embodiments mentioned above, the metallic base body has a porous surface, which is covered with the coating that contains SiO₂ or silicate. In other words, the accessible pores of the porous surface of the metallic base body contain the aforementioned nanoparticles or are coated with a polymer matrix containing the nanoparticles or the accessible pores are covered by a film of SiO₂ or silicate. The contact surface with the biological system may be enlarged in this way and the effects on apoptosis can be enhanced as desired according to the present disclosure.

The basic metallic structure is preferably made of magnesium, a biocorridible magnesium alloy, pure iron, a biocorridible iron alloy, a biocorridible tungsten alloy, a biocorridible zinc alloy or a biocorridible molybdenum alloy. The aforementioned biocorridible metallic materials are at least largely chemically inert with respect to SiO₂ and silicates so no negative effect on degradation of the stent need be expected.

For purposes of the present disclosure, biocorridible refers to alloys and elements in which a degradation/conversion takes place in a physiological environment so that the part of the implant made of this material is no longer present at all or at least is not predominately present.

For purposes of the present disclosure, the terms magnesium alloy, iron alloy, zinc alloy, molybdenum alloy and tungsten alloy refer to a metallic structure whose main component is magnesium, iron, zinc, molybdenum or tungsten. The main component is the alloy component that constitutes the largest amount by weight of the alloy. The amount of the main component is preferably more than 50 wt %, in particular, more than 70 wt %. The composition of the alloy is to be selected so that it is biocorridible. Synthetic plasma such as that specified according to EN ISO 10993-15:2000 for biocorrosion testing (composition NaCl 6.8 g/L, CaCl2 0.2 g/L, KCl 0.4 g/L, MgSO₄ 0.1 g/L, NaHCO₃ 2.2 g/L, Na₂HPO₄ 0.126 g/L, NaH₂PO₄ 0.026 g/L) is used as the test medium for testing the corrosion behavior of an alloy being considered. A sample of the alloy to be tested is stored at 37° C. in a sealed sample container with a defined amount of the test medium. The samples are removed at intervals (based on the expected corrosion behavior) of a few hours up to several months and tested for traces of corrosion by known methods. The synthetic plasma according to EN ISO 10993-15:2000 corresponds to a medium resembling blood and thus constitutes a possibility of reproducibly simulating a physiological environment according to the present disclosure.

A second aspect of the invention provides a method for producing a coating that contains SiO₂ on a stent of a metallic base body. In one exemplary embodiment, the method comprises the steps of:
(i) providing a stent made of a metallic base body;
(ii) contacting a surface of the base body with an aqueous colloidal dispersion containing amorphous SiO₂ particles with an average particle size in the range of 5-75 nm; and
(iii) at the same time or following step (ii), thermally treating the stent at least in the area of the contact surface, forming the coating that contains SiO₂.

Coatings containing SiO₂ can be created on stents of metallic base bodies in an especially simple manner by using the method described herein.

An aqueous colloidal dispersion containing only the amorphous SiO₂ particles is preferably used, and step (iii) is performed so that an SiO₂ film is formed. According to this exemplary embodiment, there is thus an agglomeration of the SiO₂ particles contained in the dispersion on the surface of the implant, forming an SiO₂ film.

Alternatively, in step (ii) an aqueous colloidal dispersion containing polyurethane and a polyisocyanate curing agent may be used in step (ii), and step (iii) is performed to form a polymer matrix of polyurethane with embedded SiO₂ nanoparticles. In other words, due to the presence of the polyurethane matrix, agglomeration of SiO₂ particles on the surface of the metal base body is prevented and instead SiO₂ nanoparticles are formed.

The metallic base body of the stent preferably has a porous surface, and step (ii) is performed so that the dispersion penetrates into the pores of the porous surface. In other words, an internal surface of the accessible pores is covered by an SiO₂ film and/or by a polymer matrix with SiO₂ nanoparticles.

The invention is explained in greater detail hereafter on the basis of an exemplary embodiment.

The stent of the biodegradable magnesium alloy WE43 (according to ASTM) was degreased and dried.

The following solutions/dispersions were prepared:
(A) A colloidally disperse solution of amorphous silicon dioxide particles with an average particle size of 6 nm in a concentration of 15% (obtainable with the brand name LEVASIL 500/15% from the company H.C. Starck).
(B) An approx. 39.5% polyurethane dispersion with blocked isocyanate groups as the curing agent component (obtainable under the brand name BAYHYDUR™ 5140 from the company Bayer MaterialScience).

The solutions/dispersions (A) and (B) were combined in a ratio of 1:1. Due to the blocking of the isocyanate groups, no crosslinking occurs at room temperature. The stent was sprayed with the combined solution. Then it was dried for 30 minutes at 150° C.

Three stents with the coating containing the silicate were implanted in experimental animal (domestic swine). After 14 days or 28 days, the coronary vessels were angiographed, explanted and evaluated histologically. It was found that the area of neointima formation was greatly reduced in comparison with that with the uncoated stents made of the magnesium alloy WE43.

What is claimed is:

1. A stent, comprising:
(i) a metallic base body; and
(ii) either an SiO₂-containing or silicate-containing coating or an SiO₂-containing or silicate-containing filling of a cavity.

2. The stent of claim 1, wherein the coating or filling contains nanoparticles of either SiO₂ or silicate.

3. The stent of claim 2, wherein the nanoparticles have an average particle size in the range of 5 to 75 nm.

4. The stent of claim 2, wherein the nanoparticles are embedded in a polymer matrix.

5. The stent of claim 4, wherein the matrix contains polyurethane.

6. The stent of claim 1, wherein the coating is a closed film of either SiO₂ or silicate.

7. The stent of claim 6, wherein the film of either SiO₂ or silicate has a thickness in the range of 1 to 15 μm.

8. The stent of claim 1, wherein the metallic base body has a porous surface which is covered with either the SiO₂-containing coating or the silicate-containing coating.

9. The stent of claim 1, wherein the metallic basic structure of the stent comprises a material selected from the group consisting of magnesium, a biocorridible magnesium alloy, pure iron, a biocorridible iron alloy, a biocorridible tungsten alloy, a biocorridible zinc alloy and a biocorridible molybdenum alloy.

10. A method for manufacturing an SiO₂-containing coating on a stent of a metallic base body, comprising:
(i) providing the stent of a metallic base body;
(ii) contacting a surface of the base body with an aqueous colloidal dispersion containing amorphous SiO₂ particles with an average particle size in the range of 5-75 nm; and
(iii) either simultaneously or in following step (ii), thermally treating the stent at least in the area of the contact surface, forming the SiO₂-containing coating.

11. The method of claim 10, wherein an aqueous colloidal dispersion is used in step (ii), containing only the amorphous SiO₂ particles, and step (iii) is performed in such a way that a film of SiO₂ is formed.

12. The method of claim 10, wherein an aqueous colloidal dispersion containing polyurethane and a polyisocyanate curing agent is used in step (ii), and step (iii) is performed in such a way that a polymer matrix of polyurethane in which the nanoparticles of SiO₂ are embedded is formed.

13. The method of claim 10, wherein the metallic base body of the stent has a porous surface and step (ii) is performed in such a way that the dispersion penetrates into the pores of the porous surface.
